(19) United States
(12) Patent Application Publication
(10) Pub. No.: US 2022/0312767 A1
(43) Pub. Date: Oct. 6, 2022

(54) PYRIDAZINE COMPOUNDS AND HERBICIDES

(71) Applicant: Nippon Soda Co., Ltd., Chiyoda-ku, Tokyo (JP)

(72) Inventors: Ken MIHARA, Odawara-shi (JP); Yoji IKEDA, Odawara-shi (JP); Yukina TAKI, Odawara-shi (JP); Kazushige KATO, Odawara-shi (JP)

(73) Assignee: Nippon Soda Co., Ltd., Chiyoda-ku, Tokyo (JP)

(21) Appl. No.: 17/634,126

(22) PCT Filed: Sep. 23, 2020
(86) PCT No.: PCT/JP2020/035658

§ 371 Date: Feb. 9, 2022

(30) Foreign Application Priority Data
Sep. 25, 2019 (JP) 2019-174533

(51) Int. Cl. A01N 43/58; C07D 237/16; A01P 13/00

(52) U.S. Cl. CPC A01N 43/58; C07D 237/16; A01P 13/00

(57) ABSTRACT

The present invention relates to compounds represented by formula (I) (in the formula, R¹ represents, e.g., a substituted or unsubstituted C1-6 alkyl group, R² represents a group represented by R^{a}—CO—O—CR^{b}₂— or a group represented by R^{a}O—CO—O—CR^{b}₂—, each R^{a} independently represents a substituted or unsubstituted C1-6 alkyl group, each R^{b} independently represents a hydrogen atom or a substituted or unsubstituted C1-6 alkyl group, R³ represents, e.g., a hydrogen atom, and Q represents, e.g., a substituted or unsubstituted phenyl group), or salts thereof, and herbicides comprising at least one selected therefrom as an effective component.

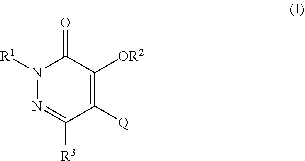

### TECHNICAL FIELD

The present invention relates to a pyridazine compound and a herbicide including the same as an active ingredient thereof.

Priority is claimed on Japanese Patent Application No. 2019-174533, filed Sep. 25, 2019, the content of which is incorporated herein by reference.

### BACKGROUND OF THE INVENTION

A herbicide may be used to control weeds in the cultivation of agricultural or horticultural crops. Various compounds have been proposed as active ingredients of herbicides.

For example, a pyridazine compound of formula (A) is disclosed in Patent Document 1.

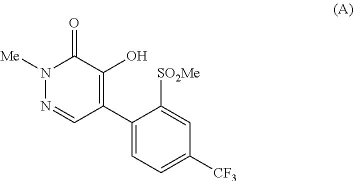 [SMILES: Cn1ncc(-c2ccc(C(F)(F)F)cc2S(C)(=O)=O)c(O)c1=O]

### DOCUMENTS OF RELATED ART

### Patent Documents

Patent Document 1: WO 2013/050421 A

### SUMMARY OF THE INVENTION

### Problems to be Solved by the Invention

It is required that a herbicide not only exhibit excellent weed control effects, but also cause fewer harmful effects on crops, fewer residual effects on the environment, and less environmental pollution.

The present invention aims to provide: a novel pyridazine compound useful as an active ingredient of a herbicide that exhibits reliable weed control effects even at a low dose, causes fewer harmful effects on crops, and has high environmental safety; and a herbicide.

### Means to Solve the Problems

The present invention encompassing the following aspects has been completed as a result of studying to solve the above-described problems.

(1) A compound of formula (I).

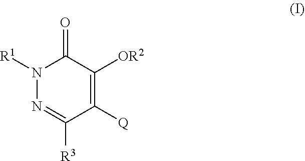

In the formula (I),

R¹ represents a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group,

R² represents a group of R^{a}—CO—O—CR^{b}₂— or a group of R^{a}O—CO—O—CR^{b}₂—,

R^{a} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,

R^{b} each independently represents a hydrogen atom or a substituted or unsubstituted C1-6 alkyl group,

R³ represents a hydrogen atom, a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group, and

Q represents a substituted or unsubstituted phenyl group or a substituted or unsubstituted naphthyl group.

(2) The compound or the salt according to (1), wherein a substituent of the phenyl group or the naphthyl group as Q is at least one selected from the group consisting of halogeno groups, substituted or unsubstituted C1-6 alkyl groups, substituted or unsubstituted C2-6 alkenyl groups, substituted or unsubstituted C2-6 alkynyl groups, a hydroxy group, substituted or unsubstituted C1-6 alkoxy groups, substituted or unsubstituted C2-6 alkenyloxy groups, substituted or unsubstituted C2-6 alkynyloxy groups, substituted or unsubstituted C1-6 alkylthio groups, substituted or unsubstituted C1-6 alkylsulfinyl groups, substituted or unsubstituted C1-6 alkylsulfonyl groups, substituted or unsubstituted C3-6 cycloalkyl groups, substituted or unsubstituted C3-6 cycloalkyloxy groups, substituted or unsubstituted phenyl groups, substituted or unsubstituted naphthyl groups, substituted or unsubstituted phenoxy groups, substituted or unsubstituted naphthoxy groups, substituted or unsubstituted phenylthio groups, substituted or unsubstituted naphthylthio groups, substituted or unsubstituted phenylsulfinyl groups, substituted or unsubstituted naphthylsulfinyl groups, substituted or unsubstituted phenylsulfonyl groups, substituted or unsubstituted naphthylsulfonyl groups, substituted or unsubstituted 5-membered or 6-membered heterocyclyl groups, substituted or unsubstituted 5-membered or 6-membered heterocyclyloxy groups, substituted or unsubstituted 5-membered or 6-membered heterocyclylthio groups, substituted or unsubstituted 5-membered or 6-membered heterocyclylsulfinyl groups, substituted or unsubstituted 5-membered or 6-membered heterocyclylsulfonyl groups, a nitro group, a cyano group, groups of R^{c}—CO—, a carboxy group, groups of R^{d}—O—CO—, groups of R^{e}R^{f}N—, groups of R^{e}R^{f}N—CO—, groups of R^{e}R^{f}N—SO₂—, groups of R^{c}—CO—O—, groups of R^{c}—CO—NH—, groups of R^{d}—SO₂—NH—, groups of R^{d}—O—CO—O—, groups of R^{d}—O—CO—NH—, groups of R^{e}R^{f}N—CO—O—, and groups of R^{e}R^{f}N—CO—NH—;

R^{c} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl groups,

R^{d} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,

R^{e} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,

R^{f} each independently represents a hydrogen atom, a substituted or unsubstituted C1-6 alkyl group, a substituted or unsubstituted phenyl group, or a substituted or unsubstituted naphthyl group,

R^{e} and R^{f} may form a divalent organic group together; and

two substituents on the phenyl group or the naphthyl group as Q may be bonded together with atoms constituting the phenyl group or the naphthyl group bonded thereby to form a substituted or unsubstituted C5-6 non-aromatic carbon ring, a substituted or unsubstituted nitrogen-containing hetero ring, a substituted or unsubstituted oxygen-containing hetero ring, or a substituted or unsubstituted oxygen and nitrogen-containing hetero ring.

(3) A herbicide including at least one selected from the group consisting of the compound and the salt thereof of (1) or (2) as an active ingredient thereof.
(4) A method for controlling monocot and/or dicot weeds on useful plants, including a step of applying the compound or the salt thereof of (1) or (2), or a herbicide including the compound on the weeds, useful plants, and/or places on which they exist.

### Effects of the Invention

The pyridazine compound according to the present invention exhibits reliable weed control effects even at a low dose, causes fewer harmful effects on crops, and has high environmental safety, and therefore is useful as an active ingredient of a herbicide. The herbicide according to the present invention can be used safely to control weeds in the cultivation of agricultural or horticultural crops.

### EMBODIMENTS FOR CARRYING OUT THE INVENTION

A pyridazine compound according to the present invention (hereinafter, may be abbreviated as “the compound according to the present invention” simply) is a compound of formula (I) (may be referred to as the compound (I)) and a salt of the compound (I). The term “compound (I)” encompasses hydrates, various solvates, crystal polymorphs, and the like. Stereoisomers or tautomers based on asymmetric carbons, double bonds or the like, of the compound (I) may present. All of these isomers and mixtures thereof are encompassed in the technical scope of the present invention.

In the present specification, the term “unsubstituted” refers to a group consisting of a mother nucleus. In the case where only the name of a group serving as a mother nucleus is provided, this refers to “unsubstituted” unless specifically indicated otherwise.

On the other hand, the term “substituted” means that any hydrogen atom of a group serving as a mother nucleus is substituted with a group having a structure that is identical to or different from that of the mother nucleus. Thus, a “substituent” is another group bound to a group serving as the mother nucleus. The number of substituents may be one or more. Two or more substituents may be identical to or different from each other.

The term “C1-6” means that the number of carbon atoms constituting a group serving as a mother nucleus is 1 to 6. The number of carbon atoms does not include the number of carbon atoms constituting a substituent. For example, a butyl group having an ethoxy group as a substituent is classified as a C2 alkoxy C4 alkyl group.

There are no particular limitations on a “substituent” provided that the substituent is chemically acceptable and achieves the effects of the present invention. Examples of a group that can be a “substituent” include the following groups:

C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group;

C2-6 alkenyl groups such as a vinyl group, a 1-propenyl group, a 2-propenyl group (allyl group), a 1-butenyl group, a 2-butenyl group, a 3-butenyl group, a 1-methyl-2-propenyl group, and a 2-methyl-2-propenyl group;

C2-6 alkynyl groups such as an ethynyl group, a 1-propynyl group, a 2-propynyl group, a 1-butynyl group, a 2-butynyl group, a 3-butynyl group, and a 1-methyl-2-propynyl group;

C3-6 cycloalkyl groups such as a cyclopropyl group, a cyclobutyl group, a cyclopentyl group, and a cyclohexyl group;

a phenyl group, a naphthyl group;

phenyl C1-6 alkyl groups such as a benzyl group, and a phenethyl group;

3- to 6-membered heterocyclyl groups;

3- to 6-membered heterocyclyl C1-6 alkyl groups;

a hydroxy group;

C1-6 alkoxy groups such as a methoxy group, an ethoxy group, a n-propoxy group, an i-propoxy group, a n-butoxy group, a s-butoxy group, an i-butoxy group, and a t-butoxy group;

C2-6 alkenyloxy groups such as a vinyloxy group, an allyloxy group, a propenyloxy group, and a butenyloxy group;

C2-6 alkynyloxy groups such as an ethynyloxy group, and a propargyloxy group;

a phenoxy group, a naphthoxy group;

C6-10 aryl C1-6 alkoxy groups such as a benzyloxy group, and a phenethyloxy group;

5-membered or 6-membered heteroaryloxy groups such as a thiazolyloxy group and a pyridyloxy group;

5-membered or 6-membered heteroaryl C1-6 alkyloxy groups such as a thiazolylmethyloxy group and a pyridylmethyloxy group;

a formyl group;

C1-6 alkylcarbonyl groups such as an acetyl group and a propionyl group;

a formyloxy group;

C1-6 alkylcarbonyloxy groups such as an acetyloxy group and a propionyloxy group;

a benzoyl group;

C1-6 alkoxycarbonyl groups such as a methoxycarbonyl group, an ethoxycarbonyl group, a n-propoxycarbonyl group, an i-propoxycarbonyl group, a n-butoxycarbonyl group, and a t-butoxycarbonyl group;

C1-6 alkoxycarbonyloxy groups such as a methoxycarbonyloxy group, an ethoxycarbonyloxy group, a n-propoxycarbonyloxy group, an i-propoxycarbonyloxy group, a n-butoxycarbonyloxy group, and a t-butoxycarbonyloxy group;

a carboxyl group;

halogeno groups such as a fluoro group, a chloro group, a bromo group, and an iodo group;

C1-6 haloalkyl groups such as a chloromethyl group, a chloroethyl group, a trifluoromethyl group, a 1,2-dichloro-n-propyl group, a 2,2,2-trifluoroethyl group, and a 1-fluoro-n-butyl group;

C2-6 haloalkenyl groups such as a 2-chloro-1-propenyl group, and a 2-fluoro-1-butenyl group;

C2-6 haloalkynyl groups such as a 4,4-dichloro-1-butynyl group, a 4-fluoro-1-pentynyl group, and a 5-bromo-2-pentynyl group;

C1-6 haloalkoxy groups such as a trifluoromethoxy group, a 2,2,2-trifluoroethoxy group, and a 2,3-dichlorobutoxy group;

C2-6 haloalkenyloxy groups such as a 2-chloropropenyloxy group and a 3-bromobutenyloxy group;

C1-6 haloalkylcarbonyl groups such as a chloroacetyl group, a trifluoroacetyl group, and a trichloroacetyl group;

an amino group;

C1-6 alkyl-substituted amino groups such as a methylamino group, a dimethylamino group, and a diethylamino group;

an anilino group, and a naphthylamino group;

phenyl C1-6 alkylamino groups such as a benzylamino group, and a phenethylamino group;

a formylamino group;

C1-6 alkylcarbonylamino groups such as an acetylamino group, a propanoylamino group, a butyrylamino group, and an i-propylcarbonylamino group;

C1-6 alkoxycarbonylamino groups such as a methoxycarbonylamino group, an ethoxycarbonylamino group, a n-propoxycarbonylamino group, and an i-propoxycarbonylamino group;

unsubstituted or substituted aminocarbonyl groups such as an aminocarbonyl group, a dimethylaminocarbonyl group, a phenylaminocarbonyl group, and a N-phenyl-N-methylaminocarbonyl group;

imino C1-6 alkyl groups such as an iminomethyl group, a (1-imino)ethyl group, and a (1-imino)-n-propyl group;

substituted or unsubstituted N-hydroxyimino C1-6 alkyl groups such as a N-hydroxy-iminomethyl group, a (1-(N-hydroxy)-imino)ethyl group, a (1-(N-hydroxy)-imino)propyl group, a N-methoxy-iminomethyl group, and a (1-(N-methoxy)-imino)ethyl group;

an aminocarbonyloxy group;

C1-6 alkyl-substituted aminocarbonyloxy groups such as an ethylaminocarbonyloxy group, and a dimethylaminocarbonyloxy group;

a mercapto group;

C1-6 alkylthio groups such as a methylthio group, an ethylthio group, a n-propylthio group, an i-propylthio group, a n-butylthio group, an i-butylthio group, a s-butylthio group, and a t-butylthio group;

C1-6 haloalkylthio groups such as a trifluoromethylthio group, and a 2,2,2-trifluoroethylthio group;

a phenylthio group;

5-membered or 6-membered heteroarylthio groups such as a thiazolylthio group, and a pyridylthio group;

C1-6 alkylsulfinyl groups such as a methylsulfinyl group, an ethylsulfinyl group, and a t-butylsulfinyl group;

C1-6 haloalkylsulfinyl groups such as a trifluoromethylsulfinyl group, and a 2,2,2-trifluoroethylsulfinyl group;

a phenylsulfinyl group;

5-membered or 6-membered heteroarylsulfinyl groups such as a thiazolylsulfinyl group, and a pyridylsulfinyl group;

C1-6 alkylsulfonyl groups such as a methylsulfonyl group, an ethylsulfonyl group, and a t-butylsulfonyl group;

C1-6 haloalkylsulfonyl groups such as a trifluoromethylsulfonyl group, and a 2,2,2-trifluoroethylsulfonyl group;

a phenylsulfonyl group;

5-membered or 6-membered heteroarylsulfonyl groups such as a thiazolylsulfonyl group, and a pyridylsulfonyl group;

C1-6 alkylsulfonyloxy groups such as a methylsulfonyloxy group, an ethylsulfonyloxy group, and a t-butylsulfonyloxy group;

C1-6 haloalkylsulfonyloxy groups such as a trifluoromethylsulfonyloxy group, and a 2,2,2-trifluoroethylsulfonyloxy group;

tri-C1-6 alkyl-substituted silyl groups such as a trimethylsilyl group, a triethylsilyl group, and a t-butyldimethylsilyl group;

a triphenylsilyl group;

a pentafluorosulfanyl group;

a cyano group; and a nitro group.

In addition, any hydrogen atom in the “substituent” may also be substituted with another group having a different structure. Examples of the “substituent” include C1-6 alkyl groups, C1-6 haloalkyl groups, C1-6 alkoxy groups, C1-6 haloalkoxy groups, halogeno groups, a cyano group, and a nitro group.

The term “3- to 6-membered heterocyclyl group” refers to a 3-membered ring group, 4-membered ring group, 5-membered ring group, or 6-membered ring group, including, as (an) atom(s) constituting the ring, 1 to 4 hetero atom(s) selected from the group consisting of a nitrogen atom, an oxygen atom and a sulfur atom. The heterocyclyl group may be monocyclic or polycyclic. If at least one ring of a polycyclic heterocyclyl group is a hetero ring, remaining rings thereof may be any of saturated alicyclic rings, unsaturated alicyclic rings and aromatic rings. Examples of the “3- to 6-membered heterocyclyl group” include 3- to 6-membered saturated heterocyclyl groups, 5-membered or 6-membered heteroaryl groups, and 5-membered or 6-membered unsaturated heterocyclyl groups.

Examples of the 3- to 6-membered saturated heterocyclyl group include an aziridinyl group, an epoxy group, an azetidinyl group, a pyrrolidinyl group, a tetrahydrofuranyl group, a dioxolanyl group, a tetrahydropyranyl group, a piperidyl group, a piperazinyl group, a morpholinyl group, and a dioxanyl group.

Examples of the 5-membered or 6-membered unsaturated heterocyclyl group include a pyrrolynyl group, a dihydrofuranyl group, an imidazolynyl group, a pyrazolynyl group, an oxazolynyl group, an isoxazolynyl group, a thiazolynyl group, an isothiazolynyl group, a dihydropyranyl group, a dihydrooxazinyl group, and a dihydroisoxazolyl group.

Examples of the 5-membered heteroaryl groups include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

Examples of the 6-membered heteroaryl group include a pyridyl group, a pyrazinyl group, a pyrimidinyl group, a pyridazinyl group, and a triazinyl group.

### [R¹]

R¹ represents a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group.

The “C1-6 alkyl group” as R¹ may be a linear chain or a branched chain. Examples of the “C1-6 alkyl group” as R¹ include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, and an i-hexyl group.

Examples of the “C2-6 alkenyl group” as R¹ include a vinyl group, a 1-propenyl group, a 2-propenyl group, a 1-butenyl group, a 2-butenyl group, a 3-butenyl group, a 1-methyl-2-propenyl group, a 2-methyl-2-propenyl group, a 1-pentenyl group, a 2-pentenyl group, a 3-pentenyl group, a 4-pentenyl group, a 1-methyl-2-butenyl group, a 2-methyl-2-butenyl group, a 1-hexenyl group, a 2-hexenyl group, a 3-hexenyl group, a 4-hexenyl group, and a 5-hexenyl group.

Examples of the “C2-6 alkynyl group” as R¹ include an ethynyl group, a 1-propynyl group, a 2-propynyl group, a 1-butynyl group, a 2-butynyl group, a 3-butynyl group, a 1-methyl-2-propynyl group, a 2-methyl-3-butynyl group, a 1-pentynyl group, a 2-pentynyl group, a 3-pentynyl group, a 4-pentynyl group, a 1-methyl-2-butynyl group, a 2-methyl-3-pentynyl group, a 1-hexynyl group, and a 1,1-dimethyl-2-butynyl group.

Preferable examples of a substituent on the “C1-6 alkyl group”, “C2-6 alkenyl group”, or “C2-6 alkynyl group” as R¹ include: halogeno groups such as a fluoro group, a chloro group, a bromo group, and an iodo group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

Examples of the “C3-6 cycloalkyl group” as R¹ include: a cyclopropyl group, a cyclobutyl group, a cyclopentyl group, and a cyclohexyl group.

Preferable examples of a substituent on the “C3-6 cycloalkyl group” as R¹ include: halogeno groups such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

In the present invention, R¹ preferably represents a substituted or unsubstituted C1-6 alkyl group.

### [R²]

R² represents a group of R^{a}—CO—O—CR^{b}₂— or a group of R^{a}O—CO—O—CR^{b}₂—.

In the “group of R^{a}—CO—O—CR^{b}₂—” and the “group of R^{a}O—CO—O—CR^{b}₂—” as R², R^{a} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group.

Examples of the “C1-6 alkyl group” as R^{a} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, and an i-hexyl group.

Examples of the “C2-6 alkenyl group” as R^{a} include a vinyl group and a 1-propenyl group.

Examples of the “C2-6 alkynyl group” as R^{a} include an ethynyl group and a 1-propynyl group.

Preferable examples of a substituent on the “C1-6 alkyl group”, “C2-6 alkenyl group” or “C2-6 alkynyl group” as R^{a} include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C3-6 cycloalkyl groups such as a cyclopropyl group, a cyclobutyl group, a cyclopentyl group, and a cyclohexyl group; a phenyl group and a naphthyl group; halogeno group-substituted, C1-6 haloalkyl group-substituted, or C1-6 haloalkoxy group-substituted phenyl groups, such as a 4-chlorophenyl group, a 4-trifluoromethylphenyl group, and a 4-trifluoromethoxyphenyl group; halogeno group-substituted, C1-6 haloalkyl group-substituted or C1-6 haloalkoxy group-substituted naphthyl groups; and a cyano group.

Examples of the “C3-6 cycloalkyl group” as R^{a} include a cyclopropyl group, a cyclobutyl group, a cyclopentyl group, and a cyclohexyl group.

The “5-membered or 6-membered heterocyclyl group” as R^{a} is a 5-membered ring group or 6-membered ring group, including, as (an) atom(s) constituting the ring, 1, 2, 3 or 4 hetero atom(s) selected from the group consisting of a nitrogen atom, an oxygen atom and a sulfur atom. In the case where at least two hetero atoms are included, the hetero atoms may be identical to or different from each other. Examples of the “5-membered or 6-membered heterocyclyl group” include 5-membered or 6-membered saturated heterocyclyl groups, 5-membered or 6-membered heteroaryl groups, and 5-membered or 6-membered unsaturated heterocyclyl groups.

Examples of the 5-membered or 6-membered saturated heterocyclyl groups include a pyrrolidinyl group, a tetrahydrofuranyl group, a dioxolanyl group, a tetrahydropyranyl group, a piperidyl group, a piperazinyl group, a morpholinyl group, and a dioxanyl group.

Examples of the 5-membered heteroaryl groups include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

Examples of the 6-membered heteroaryl groups include a pyridyl group, a pyrazinyl group, a pyrimidinyl group, a pyridazinyl group, and a triazinyl group.

Examples of the 5-membered unsaturated heterocyclyl groups include a pyrrolynyl group, a dihydrofuranyl group, an imidazolynyl group, a pyrazolynyl group, an oxazolynyl group, an isoxazolynyl group, a thiazolynyl group, and an isothiazolynyl group.

Examples of the 6-membered unsaturated heterocyclyl group include a dihydropyranyl group, and a dihydrooxazinyl group.

Preferable examples of a substituent on the “C3-6 cycloalkyl group”, “phenyl group”, “naphthyl group”, or “5-membered or 6-membered heterocyclyl group” as R^{a} include: halogeno groups such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; a phenyl group, a naphthyl group; halogeno group-substituted, C1-6 haloalkyl group-substituted, or C1-6 haloalkoxy group-substituted phenyl groups such as a 4-chlorophenyl group, a 4-trifluoromethylphenyl group, and a 4-trifluoromethoxyphenyl group; halogeno group-substituted, C1-6 haloalkyl group-substituted, or C1-6 haloalkoxy group-substituted naphthyl groups; and a cyano group.

In the “group of R^{a}—CO—O—CR^{b}₂—” and the “group of R^{a}O—CO—O—CR^{b}₂—” as R², R^{b} each independently represents a hydrogen atom or a substituted or unsubstituted C1-6 alkyl group.

Specific examples of the “C1-6 alkyl group” as R^{b} include the same groups as those mentioned as R^{a}. Specific examples of a substituent on the “C1-6 alkyl group” as R^{b} include the same groups as those mentioned as R^{a}.

Specific examples of the “group of R^{a}—CO—O—CR^{b}₂—” as R² include an acetoxymethyl group, an isopropylcarbonyloxymethyl group, a 1-acetoxyethyl group, and a 1-isopropylcarbonyloxyethyl group.

Specific examples of the “group of R^{a}O—CO—O—CR^{b}₂—” as R² include a methoxycarbonyloxymethyl group, an isopropyloxycarbonyloxymethyl group, a 1-(methyloxycarbonyloxy)ethyl group, and a 1-(isopropyloxycarbonyloxy)ethyl group.

In the present invention, R^{a} preferably represents a substituted or unsubstituted C1-6 alkyl group.

R^{b} preferably represents a hydrogen atom, or a substituted or unsubstituted C1-6 alkyl group.

### [R³]

R³ represents a hydrogen atom, a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group.

Specific examples of the substituted or unsubstituted C1-6 alkyl group, the substituted or unsubstituted C2-6 alkenyl group, the substituted or unsubstituted C2-6 alkynyl group, or the substituted or unsubstituted C3-6 cycloalkyl group as R³ include the same groups as those mentioned as R¹.

In the present invention, R³ preferably represents a hydrogen atom, or a substituted or unsubstituted C1-6 alkyl group, and more preferably represents a hydrogen atom.

### [Q]

Q represents a substituted or unsubstituted phenyl group, or a substituted or unsubstituted naphthyl group.

A substituent on the “phenyl group” or “naphthyl group” as Q (may be referred to as substituent (X)) is at least one selected from the group consisting of halogeno groups, substituted or unsubstituted C1-6 alkyl groups, substituted or unsubstituted C2-6 alkenyl groups, substituted or unsubstituted C2-6 alkynyl groups, a hydroxy group, substituted or unsubstituted C1-6 alkoxy groups, substituted or unsubstituted C2-6 alkenyloxy groups, substituted or unsubstituted C2-6 alkynyloxy groups, substituted or unsubstituted C1-6 alkylthio groups, substituted or unsubstituted C1-6 alkylsulfinyl groups, substituted or unsubstituted C1-6 alkylsulfonyl groups, substituted or unsubstituted C3-6 cycloalkyl groups, substituted or unsubstituted C3-6 cycloalkyloxy groups, substituted or unsubstituted phenyl groups, substituted or unsubstituted naphthyl groups, substituted or unsubstituted phenoxy groups, substituted or unsubstituted naphthoxy groups, substituted or unsubstituted phenylthio groups, substituted or unsubstituted naphthylthio groups, substituted or unsubstituted phenylsulfinyl groups, substituted or unsubstituted naphthylsulfinyl groups, substituted or unsubstituted phenylsulfonyl groups, substituted or unsubstituted naphthylsulfonyl groups, substituted or unsubstituted 5-membered or 6-membered heterocyclyl groups, substituted or unsubstituted 5-membered or 6-membered heterocyclyloxy groups, substituted or unsubstituted 5-membered or 6-membered heterocyclylthio groups, substituted or unsubstituted 5-membered or 6-membered heterocyclylsulfinyl groups, substituted or unsubstituted 5-membered or 6-membered heterocyclylsulfonyl groups, a nitro group, a cyano group, groups of R^{e}—CO—, a carboxy group, groups of R^{d}—O—CO—, groups of R^{e}R^{f}N—, groups of R^{e}R^{f}N—CO—, groups of R^{e}R^{f}N—SO₂—, groups of R^{e}—CO—O—, groups of R^{e}—CO—NH—, groups of R^{d}—SO₂—NH—, groups of R^{d}—O—CO—O—, groups of R^{d}—O—CO—NH—, groups of R^{e}R^{f}N—CO—O—, and groups of R^{e}R^{f}N—CO—NH—.

Examples of the “halogeno group” as X include a fluoro group, a chloro group, a bromo group, and an iodo group.

The “C1-6 alkyl group” as X may be a linear chain or a branched chain. Examples of the C1-6 alkyl group as X include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, and an i-hexyl group.

Examples of the “C2-6 alkenyl group” as X include a vinyl group, a 1-propenyl group, a 2-propenyl group, a 1-butenyl group, a 2-butenyl group, a 3-butenyl group, a 1-methyl-2-propenyl group, a 2-methyl-2-propenyl group, a 1-pentenyl group, a 2-pentenyl group, a 3-pentenyl group, a 4-pentenyl group, a 1-methyl-2-butenyl group, a 2-methyl-2-butenyl group, a 1-hexenyl group, a 2-hexenyl group, a 3-hexenyl group, a 4-hexenyl group, and a 5-hexenyl group.

Examples of the “C2-6 alkynyl group” as X include an ethynyl group, a 1-propynyl group, a 2-propynyl group, a 1-butynyl group, a 2-butynyl group, a 3-butynyl group, a 1-methyl-2-propynyl group, a 2-methyl-3-butynyl group, a 1-pentynyl group, a 2-pentynyl group, a 3-pentynyl group, a 4-pentynyl group, a 1-methyl-2-butynyl group, a 2-methyl-3-pentynyl group, a 1-hexynyl group, and a 1,1-dimethyl-2-butynyl group.

Examples of the “C1-6 alkoxy group” as X include a methoxy group, an ethoxy group, a n-propoxy group, a n-butoxy group, a n-pentyloxy group, a n-hexyloxy group, an i-propoxy group, an i-butoxy group, a s-butoxy group, a t-butoxy group, and an i-hexyloxy group.

Examples of the “C2-6 alkenyloxy group” as X include a vinyloxy group, an allyloxy group, a propenyloxy group, and a butenyloxy group.

Examples of the “C2-6 alkynyloxy group” as X include an ethynyloxy group, and a propargyloxy group.

Examples of the “C1-6 alkylthio group” as X include a methylthio group, an ethylthio group, a n-propylthio group, a n-butylthio group, a n-pentylthio group, a n-hexylthio group, and an i-propylthio group.

Examples of the “C1-6 alkylsulfinyl group” as X include a methylsulfinyl group, an ethylsulfinyl group, and a t-butylsulfinyl group.

Examples of the “C1-6 alkylsulfonyl group” as X include a methylsulfonyl group, an ethylsulfonyl group, and a t-butylsulfonyl group.

Preferable examples of a substituent on the “C1-6 alkyl group”, “C2-6 alkenyl group”, “C2-6 alkynyl group”, “C1-6 alkoxy group”, “C2-6 alkenyloxy group”, “C2-6 alkynyloxy group”, “C1-6 alkylthio group”, “C1-6 alkylsulfinyl group”, or “C1-6 alkylsulfonyl group” as X include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; a phenyl group, a naphthyl group; halogeno group-substituted, C1-6 haloalkyl group-substituted, or C1-6 haloalkoxy group-substituted phenyl groups, such as a 4-chlorophenyl group, a 4-trifluoromethylphenyl group, and a 4-trifluoromethoxyphenyl group; halogeno group-substituted, C1-6 haloalkyl group-substituted, or C1-6 haloalkoxy group-substituted naphthyl groups; and a cyano group.

Examples of the “C3-6 cycloalkyl group” as X include a cyclopropyl group, a cyclobutyl group, a cyclopentyl group, and a cyclohexyl group.

Examples of the “C3-6 cycloalkyloxy group” as X include a cyclopropyloxy group, a cyclobutyloxy group, a cyclopentyloxy group, a cyclohexyloxy group and a cycloheptyloxy group.

The “5-membered or 6-membered heterocyclyl group” as X is a 5-membered ring group or 6-membered ring group, including, as (an) atom(s) constituting the ring, 1, 2, 3 or 4 hetero atom(s) selected from the group consisting of a nitrogen atom, an oxygen atom and a sulfur atom. In the case where at least two hetero atoms are included, the hetero atoms may be identical to or different from each other. Examples of the “5-membered or 6-membered heterocyclyl group” include 5-membered or 6-membered saturated heterocyclyl groups, 5-membered or 6-membered unsaturated heterocyclyl groups, and 5-membered or 6-membered heteroaryl group.

Examples of the 5-membered saturated heterocyclyl group include a pyrrolidinyl group, a tetrahydrofuranyl group, and a dioxolanyl group.

Examples of the 6-membered saturated heterocyclyl group include a tetrahydropyranyl group, a piperidyl group, a piperazinyl group, a morpholinyl group, and a dioxanyl group.

Examples of the 5-membered unsaturated heterocyclyl group include a pyrrolynyl group, a dihydrofuranyl group, an imidazolynyl group, a pyrazolynyl group, an oxazolynyl group, an isoxazolynyl group, a thiazolynyl group, an isothiazolynyl group, and a dihydroisoxazolyl group.

Examples of the 6-membered unsaturated heterocyclyl group include a dihydropyranyl group and a dihydrooxazinyl group.

Examples of the 5-membered heteroaryl group include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

Examples of the 6-membered heteroaryl group include a pyridyl group, a pyrazinyl group, a pyrimidinyl group, a pyridazinyl group, and a triazinyl group.

The “5-membered or 6-membered heterocyclyloxy group” as X has a structure in which a 5-membered or 6-membered heterocyclyl group and an oxy group are bonded. Specific examples thereof include a thiazolyloxy group and a pyridyloxy group.

Examples of the “5-membered or 6-membered heterocyclylthio group” as X include a thiazolylthio group and a pyridylthio group.

Examples of the “5-membered or 6-membered heterocyclylsulfinyl group” as X include a thiazolylsulfinyl group and a pyridylsulfinyl group.

Examples of the “5-membered or 6-membered heterocyclylsulfonyl group” as X include a thiazolylsulfonyl group and a pyridylsulfonyl group.

Preferable examples of a substituent on the “C3-6 cycloalkyl group”, “C3-6 cycloalkyloxy group”, “phenyl group”, “naphthyl group”, “phenoxy group”, “naphthoxy group”, “phenylthio group”, “naphthylthio group”, “phenylsulfinyl group”, “naphthylsulfinyl group”, “phenylsulfonyl group”, “naphthylsulfonyl group”, “5-membered or 6-membered heterocyclyl group”, “5-membered or 6-membered heterocyclyloxy group”, “5-membered or 6-membered heterocyclylthio group”, “5-membered or 6-membered heterocyclylsulfinyl group”, or “5-membered or 6-membered heterocyclylsulfonyl group” as X include: halogeno groups such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; CJ-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

In the “group of R^{c}—CO—”, “group of R^{c}—CO—O—” or “group of R^{c}—CO—NH—” as X, R^{c} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group.

Examples of the “C1-6 alkyl group” as R^{c} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, and an i-hexyl group.

Examples of the “C2-6 alkenyl group” as R^{c} include a vinyl group and a 1-propenyl group.

Examples of the “C2-6 alkynyl group” as R^{c} include an ethynyl group, and a 1-propynyl group.

Preferable examples of a substituent on the “C1-6 alkyl group”, “C2-6 alkenyl group”, or “C2-6 alkynyl group” as R^{c} include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C3-6 cycloalkyl groups such as a cyclopropyl group, a cyclobutyl group, a cyclopentyl group, and a cyclohexyl group; a phenyl group, a naphthyl group; halogeno group-substituted, C1-6 haloalkyl group-substituted, or C1-6 haloalkoxy group-substituted phenyl groups such as a 4-chlorophenyl group, a 4-trifluoromethylphenyl group, and a 4-trifluoromethoxyphenyl group; halogeno group-substituted, C1-6 haloalkyl group-substituted, or C1-6 haloalkoxy group-substituted naphthyl groups; and a cyano group.

Examples of the “C3-6 cycloalkyl group” as R^{c} include a cyclopropyl group, a cyclobutyl group, a cyclopentyl group, and a cyclohexyl group.

The “5-membered or 6-membered heterocyclyl group” as R^{c} is a 5-membered ring group or 6-membered ring group, including, as (an) atom(s) constituting the ring, 1, 2, 3 or 4 hetero atom(s) selected from the group consisting of a nitrogen atom, an oxygen atom and a sulfur atom. In the case where at least two hetero atoms are included, the hetero atoms may be identical to or different from each other. Examples of the “5-membered or 6-membered heterocyclyl group” include 5-membered or 6-membered saturated heterocyclyl groups, 5-membered or 6-membered heteroaryl groups, and 5-membered or 6-membered unsaturated heterocyclyl groups.

Examples of the 5-membered or 6-membered saturated heterocyclyl groups include a pyrrolidinyl group, a tetrahydrofuranyl group, a dioxolanyl group, a tetrahydropyranyl group, a piperidyl group, a piperazinyl group, a morpholinyl group, and a dioxanyl group.

Examples of the 5-membered unsaturated heterocyclyl groups include a pyrrolynyl group, a dihydrofuranyl group, an imidazolynyl group, a pyrazolynyl group, an oxazolynyl group, an isoxazolynyl group, a thiazolynyl group, and an isothiazolynyl group.

Examples of the 6-membered unsaturated heterocyclyl groups include a dihydropyranyl group and a dihydrooxazinyl group.

Examples of the 5-membered heteroaryl groups include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

Examples of the 6-membered heteroaryl groups include a pyridyl group, a pyrazinyl group, a pyrimidinyl group, a pyridazinyl group, and a triazinyl group.

Preferable examples of a substituent on the “C3-6 cycloalkyl group”, “phenyl group”, “naphthyl group”, or “5-membered or 6-membered heterocyclyl group” as R^{c} include: halogeno groups such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; a phenyl group, a naphthyl group; halogeno group-substituted, C1-6 haloalkyl group-substituted, or C1-6 haloalkoxy group-substituted phenyl groups, such as a 4-chlorophenyl group, 4-trifluoromethylphenyl group, and 4-trifluoromethoxyphenyl group; halogeno group-substituted, C1-6 haloalkyl group-substituted, or C1-6 haloalkoxy group-substituted naphthyl groups; and a cyano group.

Specific examples of the “groups of R^{c}—CO—” include an acetyl group, and an isopropylcarbonyl group.

Specific examples of the “groups of R^{c}—CO—O—” include an acetyloxy group.

Specific examples of the “groups of R^{c}—CO—NH—” include an acetylamino group.

In the “groups of R^{d}O—CO—”, “groups of R^{d}—SO₂—NH—”, “groups of R^{d}—O—CO—O—”, and “groups of R^{d}—O—CO—NH—” as X, R^{d} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group.

Specific examples of these groups as R^{d} include the same groups as those mentioned as R^{e}.

Specific examples of the “groups of R^{d}O—CO—” include a methoxycarbonyl group, and an isopropyloxycarbonyl group.

Specific examples of the “groups of R^{d}—SO₂—NH—” include a methanesulfonylamino group.

Specific examples of the “groups of R^{d}—O—CO—O—” include a methoxycarbonyloxy group and an ethoxycarbonyloxy group.

Specific examples of the “groups of R^{d}—O—CO—NH—” include a methoxycarbonylamino group.

In the “group of R^{e}R^{f}N—”, “group of R^{e}R^{f}N—CO—”, “group of R^{e}R^{f}N—SO₂—”, “group of R^{e}R^{f}N—CO—O—”, and “group of R^{e}R^{f}N—CO—NH—” as X, R^{e} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group, and R^{f} each independently represents a hydrogen atom, a substituted or unsubstituted C1-6 alkyl group, a substituted or unsubstituted phenyl group, or a substituted or unsubstituted naphthyl group.

Specific examples of these groups as R^{e} and R^{f} include the same groups as those mentioned as R^{e}.

R^{c} and R^{f} may form a divalent organic group together.

Examples of the formable divalent organic group include substituted or unsubstituted C2-5 alkylene groups and substituted or unsubstituted C1-3 alkyleneoxy C1-3 alkylene groups.

Examples of the “C2-5 alkylene group” include a dimethylene group, a trimethylene group, and a tetramethylene group.

Examples of the “C1-3 alkyleneoxy C1-3 alkylene group” include a dimethyleneoxydimethylene group.

Preferable examples of a substituent on the “C2-5 alkylene group” or “C1-3 alkyleneoxy C1-3 alkylene group” include: halogeno groups such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, and a t-butyl group; and C1-6 haloalkyl groups such as a chloromethyl group, a chloroethyl group, a trifluoromethyl group, a 1,2-dichloro-n-propyl group, and a 1-fluoro-n-butyl group.

Specific examples of the “groups of R^{e}R^{f}N—” include an amino group, a methylamino group, and a dimethylamino group.

Specific examples of the “groups of R^{e}R^{f}N—CO—” include a carbamoyl group, a N,N-dimethylaminocarbonyl group, a N-(i-propyl)aminocarbonyl group, and a N-(i-propyl)-N-methylaminocarbonyl group.

Specific examples of the “groups of R^{e}R^{f}N—SO₂—” include a N,N-dimethylaminosulfonyl group.

Specific examples of the “groups of R^{e}R^{f}N—CO—O—” include a carbamoyloxy group, and a N,N-dimethylaminocarbonyloxy group.

Specific examples of the “groups of R^{e}R^{f}N—CO—NH—” include a carbamoylamino group, and a N,N-dimethylaminocarbonylamino group.

Examples of the “groups of R^{e}R^{f}N—CO—NH—” in which R^{c} and R^{f} are bonded together to form a divalent organic group include an azetidine-1-carbonylcarboxyamide group, a pyrrolidine-1-carbonylcarboxyamide group, and a morpholine-4-carbonylcarboxyamide.

Two substituents on the phenyl group or the naphthyl group as Q may be bonded together with atoms constituting the phenyl group or the naphthyl group bonded thereby to form a substituted or unsubstituted C5-6 non-aromatic carbon ring, a substituted or unsubstituted nitrogen-containing hetero ring, a substituted or unsubstituted oxygen-containing hetero ring, or a substituted or unsubstituted oxygen and nitrogen-containing hetero ring.

In the present invention, a compound in which Q represents a substituted or unsubstituted phenyl group (see formula (I-1)) is preferable.

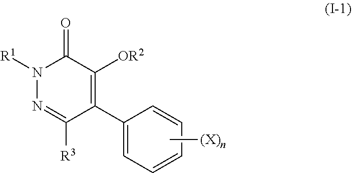

In the formula (I-1), R¹ to R³ represent the same meaning as those in the formula (I). X represents the substituent on the phenyl group mentioned above. n represents an integer of 0 to 5. In the case where n is 2 or more, X may be identical to or different from each other. In the case where n is 2 or more, two of the group X may be bonded together with atoms constituting the phenyl group or the naphthyl group bonded thereby to form a substituted or unsubstituted C5-6 non-aromatic carbon ring, a substituted or unsubstituted nitrogen-containing hetero ring, a substituted or unsubstituted oxygen-containing hetero ring, or a substituted or unsubstituted oxygen and nitrogen-containing hetero ring.

Examples of the C5-6 non-aromatic carbon ring include cycloalkanes and cycloalkenes.

Examples of the nitrogen-containing hetero ring include pyrrol, pyrroline, pyridine, dihydropyridine, pyrazole, imidazole, diazine, and triazole.

Examples of the oxygen-containing hetero ring include oxirane, furane, hydrofurane, pyrane, pyrone, dioxolane, and dioxane.

Examples of the oxygen and nitrogen-containing hetero ring include oxazole, isoxazole, and oxazine.

In the present invention, X preferably represents a halogeno group, a substituted or unsubstituted C1-6 alkyl group, a substituted or unsubstituted C2-6 alkenyl group, a substituted or unsubstituted C2-6 alkynyl group, a substituted or unsubstituted C1-6 alkoxy group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-6 cycloalkyl group, a substituted or unsubstituted phenyl group, a substituted or unsubstituted phenoxy group, a substituted or unsubstituted 5-membered heterocyclyl group, a nitro group, a cyano group, a group of R^{e}—CO—NH—, or a group of R^{e}R^{f}N—CO—NH—, and more preferably represents a halogeno group, a C1-6 alkyl group which may be substituted with a halogeno group, a C1-6 alkoxy group which may be substituted with a C1-6 alkoxy group, a substituted or unsubstituted C1-6 alkylsulfonyl group, a substituted or unsubstituted 5-membered heterocyclyl group, or a group of R^{c}—CO—NH—.

R^{e} and R^{f} may form a divalent organic group together.

The “5-membered heterocyclyl group” is a 5-membered ring group including, as (an) atom(s) constituting the ring, 1, 2, 3 or 4 hetero atom(s) selected from the group consisting of a nitrogen atom, an oxygen atom and a sulfur atom. In the case where at least two hetero atoms are included, the hetero atoms may be identical to or different from each other. Examples of the “5-membered heterocyclyl group” include 5-membered saturated heterocyclyl groups, 5-membered unsaturated heterocyclyl groups, and 5-membered heteroaryl group.

Examples of the 5-membered saturated heterocyclyl group include a pyrrolidinyl group, a tetrahydrofuranyl group, and a dioxolanyl group.

Examples of the 5-membered unsaturated heterocyclyl group include a pyrrolynyl group, a dihydrofuranyl group, an imidazolynyl group, a pyrazolynyl group, an oxazolynyl group, an isoxazolynyl group, a thiazolynyl group, an isothiazolynyl group, and a dihydroisoxazolyl group, and the 5-membered unsaturated heterocyclyl group is preferably a dihydroisoxazolyl group.

Examples of the 5-membered heteroaryl group include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

As a substituent on the “C1-6 alkyl group”, “C2-6 alkenyl group”, “C2-6 alkynyl group”, “C1-6 alkoxy group”, “C1-6 alkylthio group”, “C1-6 alkylsulfinyl group”, or “C1-6 alkylsulfonyl group”, a halogeno group, a C1-6 alkoxy group, a C1-6 haloalkoxy group, or a cyano group is preferable, and a halogeno group or a C1-6 alkoxy group is more preferable.

As a substituent on the “C3-6 cycloalkyl group”, a halogeno group, a C1-6 alkyl group, or a cyano group is preferable.

As a substituent on the “phenyl group”, “phenoxy group”, or “5-membered heterocyclyl group”, a halogeno group, a C1-6 alkyl group, a C1-6 haloalkyl group, a C1-6 alkoxy group, a C1-6 haloalkoxy group, or a cyano group is preferable.

R^{c} preferably represents a substituted or unsubstituted C1-6 alkyl group, or a substituted or unsubstituted C3-6 cycloalkyl group, and more preferably represents a C3-6 cycloalkyl group.

As a substituent on the “C1-6 alkyl group”, a halogeno group, a C1-6 alkoxy group, a C1-6 haloalkoxy group, or a cyano group is preferable.

As a substituent on the “C3-6 cycloalkyl group”, a halogeno group, a C1-6 alkyl group, or a cyano group is preferable.

R^{c} preferably represents a hydrogen atom or a substituted or unsubstituted C1-6 alkyl group, and R^{f} preferably represents a hydrogen atom or a substituted or unsubstituted C1-6 alkyl group.

As a substituent on the “C1-6 alkyl group”, a halogeno group, a C1-6 alkoxy group, a C1-6 haloalkoxy group, or a cyano group is preferable.

Examples of the divalent organic group formed by binding R^{e} and R^{f} together include substituted or unsubstituted C2-5 alkylene groups and substituted or unsubstituted C1-3 alkyleneoxy C1-3 alkylene groups.

As a substituent on the “C2-5 alkylene group” or “C1-3 alkyleneoxy C1-3 alkylene group”, a halogeno group, a C1-6 alkyl group, or a C1-6 haloalkyl group is preferable.

### [Salts]

Examples of the salts of the compound (I) include salts of alkali metals such as lithium, sodium or potassium; salts of alkaline earth metals such as calcium or magnesium; salts of transition metals such as iron or copper; ammonium salts; and salts of organic bases such as triethylamine, tributylamine, pyridine, or hydrazine.

The structure of the compound (I) or the salt of the compound (I) may be determined by NMR spectrum, IR spectrum, MS spectrum, or the like.

The compound (I) is not particularly limited by the production method thereof. The salt of the compound (I) may be obtained from the compound (I) by a conventionally-known method. For example, the compound (I) may be obtained by the method described in the below examples or the like using a compound obtained by the method disclosed in Patent Document 1 as an intermediate product thereof.

### (Reaction Scheme 1)

For example, the compound (I) may be prepared from a compound of formula (2) as shown in the following reaction scheme 1. Symbols in the formulae (2) and (2′) represent the same meanings as those in the formula (I). Xa in the formula (2′) represents a halogeno group such as a chloro group or a bromo group.

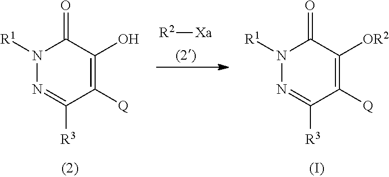

The compound of the formula (I) may be prepared by reacting the compound of the formula (2) and the compound of the formula (2′) in the presence of a preferable base (such as an inorganic base such as calcium carbonate).

In the formulae, R¹, R², R³ and Q represent the same meanings as those in the formula (I).

### (Reaction Scheme 2)

The compound of the formula (2) may be prepared from the compound of the formula (3) as shown in the following reaction scheme 2. Symbols in the formula (3) mean the same groups as those mentioned in the formula (I). R^{x} represents a lower alkyl group, such as a methyl group. Hereinafter, R^{x} represents the same meaning.

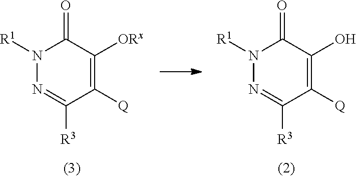

The compound of the formula (2) may be prepared by heating the compound of the formula (3) together with morpholine.

### (Reaction Scheme 3)

The compound of the formula (3) may be prepared by condensing the compound of the formula (4) and the compound of the formula (5) as shown in the following reaction scheme 3.

Symbols in the formula (4) mean the same groups as those in the formula (T). R^{y} represents a lower alkyl group, such as a methyl group or an ethyl group. Hereinafter, R^{y} represents the same meaning. In addition, R^{y} and R^{y} may be bonded together to form a 1,3,2-dioxaborolane ring. Q in the formula (5) represents the same group as that of Q in the formula (T). X^{b} represents a halogeno group.

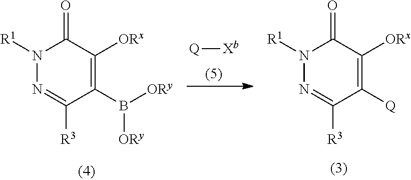

The compound of the formula (3) may be prepared by reacting the compound of the formula (4) and the compound of the formula (5) in the presence of a favorable base (such as an inorganic base such as potassium phosphate or cesium fluoride), a metal catalyst (such as a palladium catalyst such as Pd(OAc)₂) and, as needed, a ligand (such as a phosphine ligand).

The metal catalyst and the ligand may be added as a complex formed in advance (such as a palladium/phosphine complex, such as bis(triphenylphosphine)palladium dichloride or [1,1-bis(diphenylphosphino)ferrocene]palladium dichloride dichloromethane adduct).

Although Q in the compound of the formula (5) represents a substituted or unsubstituted phenyl group, or a substituted or unsubstituted naphthyl group, a substituent on the phenyl group or the naphthyl group may be approximately changed after the reaction.

### (Reaction Scheme 4)

The compound of the formula (4) may be prepared from the compound of the formula (6) as shown in the following reaction scheme 4. Symbols in the formula (6) represent the same meanings as those in the formula (1).

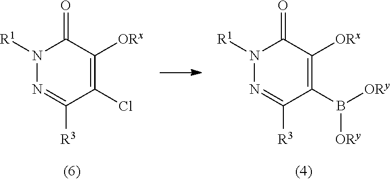

The compound of the formula (4) may be prepared by reacting the compound of formula (6) with either a boronic acid or an ester thereof, such as bis(pinacolato)diboron, in the presence of a favorable base (such as an inorganic base such as potassium phosphate or cesium fluoride), a metal catalyst (such as a palladium catalyst such as Pd₂(dba)₃, or Pd(OAc)₂), and, as needed, a ligand (such as a phosphine ligand).

The metal catalyst and the ligand may be added as a complex formed in advance (such as a palladium/phosphine complex, such as bis(triphenylphosphine)palladium dichloride or [1,1-bis(diphenylphosphino)ferrocene]palladium dichloride dichloromethane adduct).

### (Reaction Scheme 5)

The compound of the formula (6) may be prepared from the compound of the formula (7) as shown in the following reaction scheme 5. Symbols in the formula (7) represent the same groups as those in the formula (I).

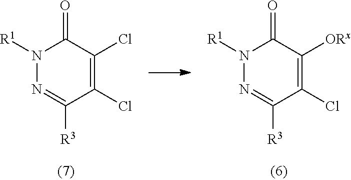

The compound of the formula (6) may be prepared by reacting the compound of the formula (7) with a favorable metal alkoxide, such as sodium methoxide.

The compound of the formula (7) may be prepared by a conventionally-known method.

### (Reaction Scheme 3A)

The compound of the formula (3) may be prepared by condensing the compound of the formula (6) and the compound of the formula (8) as shown in the following reaction scheme 3A.

Q in the formula (8) represents the same meaning as that of Q in the formula (I). R^{y} represents a lower alkyl group, such as a methyl group or an ethyl group. In addition, R^{y} and R^{y} may be bonded together to form a 1,3,2-dioxaborolane ring.

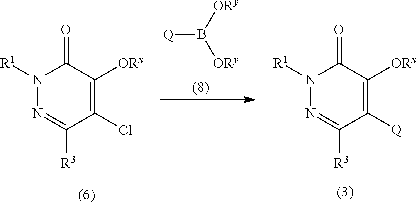

The compound of the formula (3) may be prepared by reacting the compound of formula (6) and the compound of the formula (8), in the presence of a favorable base (such as an inorganic base such as potassium phosphate or cesium fluoride), a metal catalyst (such as a palladium catalyst such as Pd(OAc)₂), and, as needed, a ligand (such as a phosphine ligand).

The metal catalyst and the ligand may be added as a complex formed in advance (such as a palladium/phosphine complex, such as bis(triphenylphosphine)palladium dichloride or [1,1-bis(diphenylphosphino)ferrocene]palladium dichloride dichloromethane adduct).

Although Q in the compound of the formula (8) represents a substituted or unsubstituted phenyl group, or a substituted or unsubstituted naphthyl group, a substituent on the phenyl group or the naphthyl group may be approximately changed after the reaction.

The compound according to the present invention exhibits high herbicidal activity under dry-field conditions by any method of soil treatment and foliage treatment.

The compound according to the present invention is effective on various field weeds and may exhibit selectivity on crops such as corn or wheat.

The compound according to the present invention may exhibit plant growth-regulating behavior such as growth-suppressing behavior against useful plants such as crops, foliage plant, or fruit-bearing trees.

The compound according to the present invention may exhibit excellent herbicidal activity against weeds in a paddy field, and may exhibit selectivity on the paddy rice.

The herbicide according to the present invention includes at least one selected from the group consisting of the compound (I) and the salts of the compound (I), as an active ingredient thereof. That is, one embodiment of the present invention is a herbicide including at least one selected from the group consisting of the compound (I) and the salts thereof, as an active ingredient thereof.

The herbicide according to the present invention exhibits high herbicidal activity under dry-field conditions by any method of a soil treatment and a foliage treatment.

The herbicide according to the present invention exhibits excellent herbicidal activity on weeds in a paddy field, such as nobie, Cyperus difforis, threeleaf arrowhead, or Schoenoplectiella hotarui, and may exhibit selectivity on paddy rice.

In addition, the herbicide according to the present invention may also be used to control weeds in a fruit farm, a lawn, a ditch next to a railway track, vacant ground, or the like.

“The method for controlling monocot and/or dicot weeds on useful plants” (weeding method) according to the present invention includes a step of applying the compound according to the present invention or a salt thereof, or the herbicide according to the present invention on useful plants, monocot and/or dicot weeds on the useful plants, and/or places on which they exist.

The soil treatment, foliage treatment, or the like may be adopted as the application method. As the soil treatment, spray treatment or mixing treatment may be conducted before or after germination of weeds.

Examples of the useful plants to be treated with the herbicide according to the present invention include crops such as cereals such as barley and wheat, cotton, brassica, sunflower, corn, rice, soybean, sugar beet, sugar cane and lawn grass.

Examples of the crops may include fruit-bearing trees, palm trees, coconut trees and additional nut trees. Additional examples thereof may include low trees of grape or fruits, fruit plants and creepers of vegetables.

Examples of the field weeds to be controlled include the following weeds.

(A) Weeds of Monocots

(1) Weeds Belonging to the Family Cyperaceae

Weeds of the genus Cyperus, such as Cyperus esculentus, Cyperus iria, Cyperus microiria, and Cyperus rotundus.

(2) Weeds Belonging to the Family Poaceae

Weeds of the genus Alopecurus, such as Alopecurus aequalis, and Alopecurus myosuroides.

Weeds of the genus Apera, such as Apera spica-venti.

Weeds of the genus Avena, such as Avena sativa.

Weeds of the genus Bromus, such as Bromus japonicus, and Bromus sterilis.

Weeds of the genus Digitaria, such as Digitaria ciliaris, and Digitaria sanguinalis.

Weeds of the genus Echinochloa, such as Echinochloa crus-galli.

Weeds of the genus Eleusine, such as Eleusine indica.

Weeds of the genus Lolium, such as Lolium multiflorum Lam.

Weeds of the genus Panicum, such as Panicum dichotomiflorum.

Weeds of the genus Poa, such as Poa annua.

Weeds of the genus Setaria, such as Setaria faberi, Setaria pumila, and Setaria viridis.

Weeds of the genus Sorghum, such as Sorghum bicolor.

Weeds of the genus Urochloa, such as Urochloa platyphylla.

(B) Weeds of Dicots

(1) Weeds Belonging to the Family Amaranthaceae

Weeds of the genus Amaranthus, such as Amaranthus blitum, Amaranthus palmeri, Amaranthus retroflexus, and Amaranthus rudis.

Weeds of the genus Chenopodium, such as Chenopodium album.

Weeds of the genus Bassia, such as Bassia scoparia.

(2) Weeds Belonging to the Family Asteraceae

Weeds of the genus Ambrosia, such as Ambrosia artemisiifolia, and Ambrosia trifida.

Weeds of the genus Conyza, such as Conyza canadensis, and Conyza sumatrensis.

Weeds of the genus Erigeron, such as Erigeron annuus.

Weeds of the genus Matricaria, such as Matricaria inodora, and Matricaria recutita.

Weeds of the genus Xanthium, such as Xanthium occidentale.

(3) Weeds Belonging to the Family Caryophyllaceae

Weeds of the genus Sagina, such as Sagina japonica.

Weeds of the genus Stellaria, such as Stellaria media.

(4) Weeds Belonging to the Family Convolvulaceae

Weeds of the genus Calystegia, such as Calystegia japonica.

Weeds of the genus Ipomoea, such as Ipomoea coccinea, Ipomoea hederacea, Ipomoea lacunosa, and Ipomoea triloba.

(5) Weeds Belonging to the Family Lamiaceae

Weeds of the genus Lamium, such as Lamium album var. barbatum, Lamium amplexicaule, and Lamium purpureum.

(6) Weeds Belonging to the Family Malvaceae

Weeds of the genus Abutilon, such as Abutilon theophrasti.

Weeds of the genus Sida, such as Sida spinosa.

(7) Weeds Belonging to the Family Plantaginaceae.

Weeds of the genus Veronica, such as Veronica persica.

(8) Weeds Belonging to the Family Polygonaceae

Weeds of the genus Fallopia, such as Fallopia convolvulus.

Weeds of the genus Persicaria, such as Persicaria lapathifolia, and Persicaria longiseta.

(9) Weeds Belonging to the Family Rubiaceae

Weeds of the genus Galium, such as Galium spurium var. echinospermon.

Examples of target weeds in a paddy field include the following weeds.

(A) Weeds of Monocotss

(1) Weeds Belonging to the Family Alismataceae

Weeds of the genus Sagittaria, such as Sagittaria pygmaea Miq., and Sagittaria trifolia.

(2) Weeds Belonging to the Family Cyperaceae

Weeds of the genus Cyperus, such as Cyperus serotinus, and Cyperus difforis.

Weeds of the genus Eleocharis, such as Eleocharis kuroguwai Ohwi.

Weeds of the genus Schoenoplectiella, such as Schoenoplectiella hotarui, and Schoenoplectiella juncoides Roxb.

Weeds of the genus Scirpus, such as Scirpus maritimus, and Scirpus nipponicus.

(3) Weeds Belonging to the Family Poaceae

Weeds of the genus Echinochloa (so-called nobie), such as Echinochloa oryzoides, and Echinochloa crus-galli.

Weeds of the genus Leersia, such as Leersia japonica.

Weeds of the genus Paspalum, such as Paspalum distichum.

(4) Weeds Belonging to the Family Pontederiaceae

Weeds of the genus Monochoria, such as Monochoria korsakowii, and Monochoria vaginalis var. plantaginea.

(B) Weeds of Dicots

(1) Weeds Belonging to the Family Apiaceae

Weeds of the genus Oenanthe, such as Oenanthe javanica.

(2) Weeds Belonging to the Family Elatinaceae

Weeds of the genus Elatine, such as Elatine triandra.

(3) Weeds Belonging to the Family Linderniaceae

Weeds of the genus Lindernia, such as Lindernia dubia subsp. major, Lindernia dubia subsp. dubia, and Lindernia procumbens.

(4) Weeds Belonging to the Family Lythraceae

Weeds of the genus Rotala, such as Rotala indica var. uliginosa.

The herbicide according to the present invention may consist of the compound according to the present invention, or may be formulated into a dosage form generally acceptable as an agrichemical, such as wettable powders, granules, powders, an emulsion, a water-soluble agent, a suspension or a floable.

A conventionally-known additive or carrier may be used to conduct the formulation.

That is, one embodiment of the present invention is a herbicide including an agrochemically acceptable solid carrier and/or liquid carrier.

In the case of a solid dosage form, a solid carrier such as a plant-derived powder, such as a soybean powder or a wheat powder, a mineral fine powder, such as a diatom earth, an apatite, a gypsum, a talc, a bentonite, a pyrophyllite, or a clay, or an organic or inorganic compound such as sodium benzoate, urea, or mirabilite may be used.

In the case of a liquid dosage form, a liquid carrier such as a petroleum fraction such as kerosene, xylene or solvent naphtha, cyclohexane, cyclohexanone, dimethylformamide, dimethylsulfoxide, alcohol, acetone, trichloroethylene, methylisobutylketone, mineral oil, vegetable oil, or water may be used.

A surfactant may be added to conduct formulation, as needed. Examples of the surfactant include: nonionic surfactants such as alkylphenyl ethers in which polyoxyethylene is added, alkyl ethers in which polyoxyethylene is added, higher fatty acid esters in which polyoxyethylene is added, sorbitan higher fatty acid esters in which polyoxyethylene is added, or tristyrylphenyl ether in which polyoxyethylene is added; alkylphenyl ether sulfuric acid ester salt in which polyoxyethylene is added, alkylnaphthalenesulfonate, polycarboxylate, ligninsulfonate, formaldehyde condensates of alkylnaphthalenesulfonate, and copolymers of isobutylene-maleic anhydride.

The amount of the active ingredient in the herbicide according to the present invention may be appropriately determined depending on the dosage form. For example, the amount of the active ingredient in wettable powders is preferably 5% by mass to 90% by mass, and more preferably 10% by mass to 85% by mass. The amount of the active ingredient in an emulsion is preferably 3% by mass to 70% by mass, and more preferably 5% by mass to 60% by mass. The amount of the active ingredient in granules is preferably 0.01% by mass to 50% by mass, and more preferably 0.05% by mass to 40% by mass.

The thus obtained wettable powders or emulsion may be diluted with water to be applied as a suspension or an emulsion having a predetermined concentration, or the granules may be applied directly to the soil before or after the germination of weeds by conducting spray treatment or mixing treatment. In the case where the herbicide according to the present invention is applied to an agricultural field, an appropriate amount of at least 0.1 g of the active ingredient per hectare may be applied.

The herbicide according to the present invention may be mixed with a conventionally-known fungicide, fungicidal active component, insecticide, insecticidal active component, acaricide, acaricidal active component, herbicide, herbicidal active component, plant growth regulator, fertilizer, or safener to be used. Particularly, the used dosage thereof can be decreased by mixing with a herbicide. In addition, not only labor-saving can be achieved, but also higher effects can be expected by synergies of mixed agents. In this case, plural conventionally-known herbicides can be mixed.

That is, one embodiment of the present invention is a herbicide further including at least one additional herbicidal active component.

In addition, one embodiment of the present invention is a herbicide further including at least one safener.

Although there is no particular limitation on the additional herbicidal active component available in the present invention, examples thereof include the following.

(a) Aryloxyphenoxypropionic acid ester-based compounds, such as clodinafop-propargyl, cyhalofop-butyl, diclofop-methyl, fenoxaprop-P-ethyl, fluazifop-P, fluazifop-P-butyl, haloxyfop-methyl, pyriphenop-sodium, propaquizafop, quizalofop-P-ethyl, and metamifop; cyclohexanedione-based compounds, such as alloxydim, butroxydim, clethodim, cycloxydim, profoxydim, sethoxydim, tepraloxydim, and tralkoxydim; phenylpyrazoline-based compounds such as pinoxaden; and other compounds that exhibit herbicidal effects by inhibiting acetyl CoA carboxylase of plants.

(b) Sulfonylurea-based compounds, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron, foramsulfuron, halosulfuron-methyl, imazosulfuron, iodosulfuron-methyl, mesosulfuron, mesosulfuron-methyl, metsulfuron-methyl, nicosulfuron, oxasulfuron, primisulfuron, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, orthosulfamuron, propyrisulfuron, flucetosulfuron, metazosulfuron, methiopyrsulfuron, monosulfuron-methyl, orsosulfuron, and iofensulfuron; imidazolinone-based compounds, such as imazapic, imazamethabenz, imazamox-ammonium, imazapyr, imazaquin, and imazethapyr; triazolopyrimidinesulfonamide-based compounds, such as cloransulam-methyl, diclosulam, florasulam, flumetsulam, metosulam, penoxsulam, pyroxsulam, and metosulfam; pyrimidinyl(thio)benzoate-based compounds, such as bispyribac-sodium, pyribenzoxim, pyriftalid, pyrithiobac-sodium, pyriminobac-methyl, and pyrimisulfan; sulfonylaminocarbonyltriazolinone-based compounds, such as flucarbazone, propoxycarbazone, and thiencarbazone-methyl; sulfonanilide-based compounds, such as triafamone; and other compounds that exhibit herbicidal effects by inhibiting acetolactate synthase (ALS) (acetohydroxyacid synthase (AHAS)) of plants.

(c) Triazine-based compounds, such as ametryn, atrazine, cyanazine, desmetryne, dimethametryn, prometon, prometryn, a propazine-based compound (propazine), CAT (simazine), simetryn, terbumeton, terbuthylazine, terbutryne, trietazine, atratone, and cybutryne; triazinone-based compounds such as hexazinone, metamitron, and metribuzin; triazolinone-based compounds, such as amicarbazone; uracil-based compounds, such as bromacil, lenacil, and terbacil; pyridazinone-based compounds, scuh as PAC (chloridazon); carbamate-based compounds, such as desmedipham, phenmedipham, and swep; urea-based compounds such as chlorobromuron, chlorotoluron, chloroxuron, dimefuron, DCMU (diuron), ethidimuron, fenuron, fluometuron, isoproturon, isouron, linuron, methabenzthiazuron, metobromuron, metoxuron, monolinuron, neburon, siduron, tebuthiuron, metobenzuron, and karbutilate; amide-based compounds, such as DCPA (propanil), and CMMP (pentanochlor); anilide-based compounds, such as cypromid; nitrile-based compounds, such as bromofenoxim, bromoxynil, and ioxynil; benzothiadiazinone-based compounds, such as bentazone; phenylpyridazine-based compounds, such as pyridate, and pyridafol; and other compounds that exhibit herbicidal effects by inhibiting photosynthesis of plants, such as methazole.

(d) Bipyridirium-based compounds such as diquat and paraquat; and other compounds that exhibit instantaneous herbicidal effects by serving as a free radical in a plant body to generate active oxygen.

(e) Diphenyl ether-based compounds, such as acifluorfen-sodium, bifenox, chloromethoxynyl (chlomethoxyfen), fluoroglycofen, fomesafen, halosafen, lactofen, oxyfluorfen, nitrofen, and ethoxyfen-ethyl; phenylpyrazole-based compounds, such as fluazolate and pyraflufen-ethyl; N-phenylphthalimide-based compounds, such as cinidon-ethyl, flumioxazin, flumiclorac-pentyl, and chlorphthalim; thiadiazole-based compounds, such as fluthiacet-methyl and thidiazimin; oxadiazole-based compounds, such as oxadiazon and oxadiargyl; triazolinone-based compounds, such as azafenidin, carfentrazone-ethyl, sulfentrazone, and bencarbazone; oxazolidindione-based compounds, such as pentoxazone; pyrimidinedione-based compounds, such as benzfendizone and butafenacil; sulfonylamide-based compounds such as saflufenacil; pyridazine-based compounds such as flufenpyr-ethyl; and other compounds that exhibit herbicidal effects by inhibiting chlorophyll biosynthesis of plants to allow photosensitized peroxidation substance to accumulate abnormally in plant bodies, such as pyrachlonil, profluazol, tiafenacil, and trifludimoxazin.

(f) Pyridazinone-based compounds, such as norflurazon and metflurazon; pyridinecarboxamide-based compounds, such as diflufenican and picolinafen; triketone-based compounds, such as mesotrione, sulcotrione, tefuryltrione, tembotrione, bicyclopyrone, and fenquinotrione; isoxazole-based compounds, such as isoxachlortole, and isoxaflutole; pyrazole-based compounds, such as benzofenap, pyrazolate (pyrazolynate), pyrazoxyfen, topramezone, pyrasulfotole, and tolpyralate; triazole-based compounds, such as ATA (amitrol)); isoxazolidinone-based compounds, such as clomazone; diphenyl ether-based compounds, such as aclonifen; and other compounds that exhibit herbicidal effects by inhibiting biosynthesis of pigment, such as carotenoid, in plants to exhibit whitening effect, such as beflubutamid, fluridone, flurochloridone, flurtamone, benzobicyclone, methoxyphenone, and ketospiradox.

(g) Glycine-based compounds, such as glyphosate, glyphosate-ammonium, glyphosate-isopropylamine, and glyphosate-trimesium (sulfosate); and other EPSP synthetase inhibitors.

(h) Glutamine synthetase inhibitors such as phosphinic acid-based compounds such as glufosinate, glufosinate-ammonium, and bialaphos (bilanafos).

Other compounds that exhibit herbicidal effects by inhibiting amino acid biosynthesis in plants.

(i) Carbamate-based compounds such as asulam; and other DHP (dihydropteroic acid) synthetase inhibitors.

(j) Dinitroaniline-based compounds, such as bethrodine (benfluralin), butralin, dinitramine, ethalfluralin, oryzalin, pendimethalin, trifluralin, nitralin, and prodiamine; phosphoramidate-based compounds such as amiprofos-methyl, and butamifos; pyridine-based compounds, such as dithiopyr and thiazopyr; benzamide-based compounds, such as propyzamide and tebutam; benzoic acid-based compounds, such as chlorthal, and TCTP (chlorthal-dimethyl); carbamate-based compounds, such as IPC (chlorpropham), propham, carbetamide, and barban; arylalanine-based compounds, such as flamprop-M, and flamprop-M-isopropyl; chloroacetamide-based compounds, such as acetochlor, alachlor, butachlor, dimethachlor, dimethenamid, dimethenamid-P, metazachlor, metolachlor, S-metolachlor, pethoxamid, pretilachlor, propachlor, propisochlor, and thenylchlor; acetamide-based compounds, such as diphenamid, napropamide, and naproanilide; oxyacetamide-based compounds, such as flufenacet, and mefenacet; tetrazolinone-based compounds, such as fentrazamide; and other compounds that exhibit herbicidal effects by inhibiting microtubule polymerization, microtubule formation, cell division, or biosynthesis of very long chain fatty acid (VLCFA) in plants, such as anilofos, indanofan, cafenstrole, piperophos, methiozolin, fenoxasulfone, pyroxasulfone, and ipfencarbazone.

(k) Nitrile-based compounds, suh as DBN (dichlobenil) and DCBN (chlorthiamid); benzamide-based compounds such as isoxaben; triazolocarboxamide-based compounds such as flupoxam; quinolinecarboxylic acid-based compounds such as quinclorac; and other compounds that exhibit herbicidal effects by inhibiting cell-wall (cellulose) synthesis, such as triaziflam, and indaziflam.

(l) Dinitrophenol-based compounds, such as DNOC, DNBP (dinoseb), and dinoterb; and other compounds that exhibit herbicidal effects by uncoupling (membrane disruption).

(m) Thiocarbamate-based compounds, such as butylate, hexylthiocarbam (cycloate), dimepiperate, EPTC, esprocarb, molinate, orbencarb, pebulate, prosulfocarb, benthiocarb (thiobencarb), tiocarbazil, triallate, vernolate, and diallate; phosphorodithioate-based compounds such as SAP (bensulide); benzofuran-based compounds, such as benfuresate and ethofumesate; chlorocarboxylic acid-based compounds, such as TCA, DPA (dalapon), and tetrapion (flupropanate); and other compounds that exhibit herbicidal effects by inhibiting lipid biosynthesis in plants.

(n) phenoxcarboxylic acid-based compounds, such as clomeprop, 2,4-PA (2,4-D), 2,4-DB, dichlorprop, MCPA, MCPB, and MCPP (mecoprop); benzoic acid-based compounds, such as chloramben, MDBA (dicamba), and TCBA (2,3,6-TBA); pyridinecarboxylic acid-based compounds, such as clopyralid, aminopyralid, fluroxypyr, picloram, triclopyr, and halauxifen; quinolinecarboxylic acid-based componds, such as quinclorac, and quinmerac; phthalamate semicarbazone-based compounds, such as NPA (naptalam) and diflufenzopyr; and other compounds that exhibit herbicidal effects by disturbing hormone behavior in plants, such as benazolin, diflufenzopyr, fluroxypyr, chlorflurenol, aminocyclopyrachlor, and DAS534.

(o) Arylaminopropionic acid-based compounds, such as flamprop M methyl/isopropyl (flamprop-isopropyl); pyrazolium-based compounds, such as difenzoquat; organic arsenic-based compounds, such as DSMA and MSMA; and other herbicides such as bromobutide, chlorflurenol, cinmethylin, cumyluron, dazomet, daimuron, methyl-dymron, etobenzanid, fosamine, oxaziclomefone, oleic acid, pelargonic acid, pyributicarb, endothall, sodium chlorate, metam, quinoclamine, cyclopyrimorate, tridiphane, and clacyfos.

Examples of the safener available in the present invention include benoxacor, cloquintocet, cloquintocet-mexyl, cyometrinil, cyprosulfamide, dichlormid, dicyclonon, dietholate, fenchlorazole, fenchlorazole-ethyl, fenclorim, flurazole, fluxofenim, furilazole, isoxadifen, isoxadifen-ethyl, mefenpyr, mefenpyr-diethyl, mephenate, naphthalic anhydride, and oxabetrinil.

### FORMULATION EXAMPLES

Although some formulation examples of the herbicide according to the present invention are shown below, the compound according to the present invention (active ingredient), additives and addition amounts thereof are not limited to the examples, and may be varied in a wide range. In the formulation examples, the term “part” means “part by mass” and the term “%” means “% by mass”.

### (Formulation Example 1) Wettable Powders

| | | |
|---|---|---|
| Compound according to the present invention | 20 | parts |
| White carbon | 20 | parts |
| Diatom earth | 52 | parts |
| Sodium alkylsulfate | 8 | parts |

The above-mentioned components were mixed uniformly and pulverized finely to obtain wettable powders including 20% of the active ingredient thereof.

### (Formulation Example 2) Emulsion

| | |
|---|---|
| Compound according to the present invention | 20 parts |
| Xylene | 55 parts |
| Dimethylformamide | 15 parts |
| Polyoxyethylenephenyl ether | 10 parts |

The above-mentioned components were mixed and dissolved to obtain an emulsion including 20% of the active ingredient thereof.

### (Formulation Example 3) Granules

| | | |
|---|---|---|
| Compound according to the present invention | 5 | parts |
| Talc | 40 | parts |
| Clay | 38 | parts |
| Bentonite | 10 | parts |
| Sodium alkylsulfate | 7 | parts |

The above-mentioned components were mixed uniformly and pulverized finely, followed by granulating the resultant to obtain granules having a diameter of 0.5 mm to 1.0 mm and including 5% of the active ingredient thereof.

Next, synthesis examples are shown. However, the present invention is not limited to the following examples.

### Example 1

### Synthesis of methyl (1-((2-methyl-5-(2-(methylsulfonyl)-4-(trifluoromethyl) phenyl)-3-oxo-2,3-dihydropyridazin-4-yl)oxy)ethyl) carbonate (Compound No. A-4)

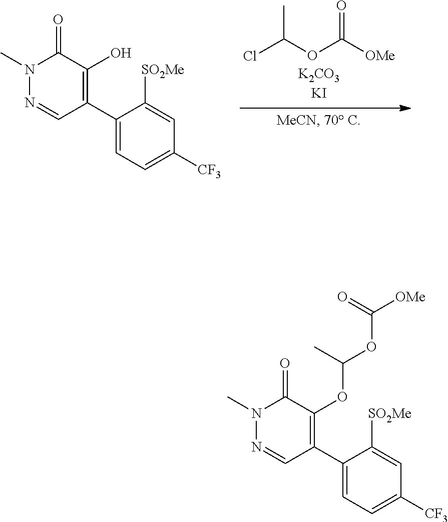

4-Hydroxy-2-methyl-5-(2-(methylsulfonyl)-4-(trifluoromethyl)phenyl)pyridazin-3(2H)-one (0.10 g), calcium carbonate (0.20 g), and potassium iodide (0.20 g) were added to acetonitrile (7.0 mL), and then the mixture was stirred at room temperature. 1-Chloroethyl methyl carbonate (0.20 mL) was added to the resultant, and the mixture was stirred at 70° C. for 4 hours.

Then, the resultant was cooled to room temperature, and then a solid material was removed therefrom by filtration, followed by concentrating the resultant filtrate under reduced pressure. The resultant concentrate was purified by silica gel column chromatography to obtain 0.09 g of a target compound.

Some examples of the compound according to the present invention prepared by the same method as that of the above-mentioned synthesis example are shown in Table 1. Substituents in the compound of formula (I-2) are shown in Table 1. In addition, the melting point thereof is also shown. Me represents a methyl group, ⁱPr represents an isopropyl group, and ^{c}Pr represents a cyclopropyl group.

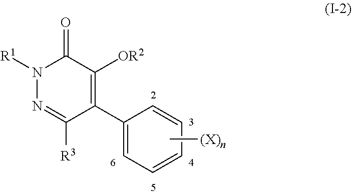

TABLE 1

| Compound | | | | | Physical |
|---|---|---|---|---|---|
| No. | R¹ | R² | R³ | (X)n | property |
| A-1 | Me | CH(Me)—O—CO—ⁱPr | H | 2-SO₂Me, 4-CF₃ | * |
| A-2 | Me | CH(Me)—O—CO—OMe | H | 2-Cl, 4-SO₂Me | m.p. 158-159° C. |
| A-3 | Me | CH₂—O—CO—OMe | H | 2-SO₂Me, 4-CF₃ | m.p. 130-131° C. |
| A-4 | Me | CH(Me)—O—CO—OMe | H | 2-SO₂Me, 4-CF₃ | m.p. 173-176° C. |
| A-5 | Me | CH₂—O—CO—ⁱPr | H | 2-SO₂Me, 4-CF₃ | m.p. 115-118° C. |
| A-6 | Me | CH(Me)—O—CO—OMe | H | 2-Me, | * |
| | | | | 3-(morpholine-4- | |
| | | | | carboxamido), | |
| | | | | 4-CF₃ | |
| A-7 | Me | CH(Me)—O—CO—OMe | H | 2-Cl, | * |
| | | | | 3-(4,5-dihydroisoxazol-3-yl), | |
| | | | | 4-SO₂Me | |
| A-8 | Me | CH(Me)—O—CO—OMe | H | 2-Cl, 3-OCH₂CH₂OMe, | * |
| | | | | 4-SO₂Me | |
| A-9 | Me | CH(Me)—O—CO—OMe | H | 2-Me, 3-NHCO^{c}Pr, 4-CF₃ | * |

Among the compounds shown in Table 1, the compounds indicated by in the column indicating the melting point were compounds having an amorphous or viscous oily property. The ¹H-NMR data thereof are shown below.

Compound A-1: ¹H-NMR (400 MHz, CDCl₃): δ 1.05 (t, 6H), 1.56 (d, 3H), 2.30-2.41 (m, 1H), 3.05 (s, 3H), 3.84 (s, 3H), 7.09 (q, 1H), 7.36 (d, 1H), 7.55 (s, 3H), 7.87 (d, 1H), 8.42 (s, 1H).

Compound A-6: ¹H-NMR (400 MHz, CDCl₃): δ 1.58 (d, 3H), 1.97 (s, 3H), 3.43-3.44 (m, 4H), 3.60-3.62 (m, 4H), 3.68 (s, 3H), 3.83 (s, 3H), 6.50 (d, 1H), 6.76-6.77 (m, 1H), 7.31 (d, 1H), 7.53 (s, 1H).

Compound A-7: ¹H-NMR (400 MHz, CDCl₃): δ 1.58 (d, 3H), 3.24 (s, 3H), 3.38-3.45 (m, 2H), 3.67 (s, 3H), 3.82 (s, 3H), 4.55-4.60 (m, 2H), 6.99-7.01 (m, 1H), 7.51-7.58 (m, 2H), 8.08 (d, 1H).

Compound A-8: ¹H-NMR (400 MHz, CDCl₃): δ 1.58 (d, 3H), 3.34 (s, 3H), 3.47 (s, 3H), 3.69 (s, 3H), 3.84-3.85 (m, 5H), 4.42-4.44 (m, 2H), 7.02-7.05 (m, 1H), 7.18 (d, 1H), 7.57 (s, 1H), 7.93 (d, 1H).

Compound A-9: ¹H-NMR (400 MHz, CDCl₃): δ 0.87-0.89 (m, 2H), 1.05-1.07 (m, 2H), 1.57-1.65 (m, 4H), 2.07 (s, 3H), 3.67 (s, 3H), 3.86 (s, 3H), 6.91 (s, 1H), 7.21 (s, 1H), 7.53-7.55 (m, 2H).

### (Evaluation of Herbicidal Effects)

Next, the usefulness of the compound according to the present invention as an active ingredient of a herbicide is shown in the following test examples.

### (Test Example 1) Foliage Treatment Test

### (1) Preparation of Test Emulsion

POA allylphenyl ether (4.1 parts by mass), POE-POP glycol (1 part by mass), POE sorbitanlaurate (0.8 parts by mass), glycerin (2.6 parts by mass), dimethylformamide (65.9 parts by mass), N-methylpyrolidone (5.1 parts by mass), cyclohexanone (15.4 parts by mass), and aromatic hydrocarbon (5.1 parts by mass) were mixed and dissolved to prepare an emulsion. The compound according to the present invention (4 mg) was dissolved in the emulsion (100 μL) to prepare a test emulsion. The term “POA” means “polyoxyalkylene”, the term “POE” means “polyoxyethylene”, and the term “POP” means “polyoxypropylene”.

### (2) Foliage Spray Treatment

150 cm² pots were filled with soil, and then seeds of Avena sativa, Matricaria chamomilla, Setaria faberi, Digitaria ciliaris, Abutilon theophrasti and Amaranthus retroflexus were sown on the surface layer of each of the pots, respectively, followed by lightly covering with soil. Then, the seeds were grown in a greenhouse. When each plant was grown to obtain a grass height of 2 cm to 4 cm, the above-mentioned test emulsion diluted to obtain a predetermined active ingredient amount was sprayed onto the leaves and stems at a spray volume of 250 L per hectare using a small sprayer.

### (3) Evaluation

Three weeks after, the fresh grass mass at an aerial part on an untreated area or a treated area was measured per weed, and the herbicidal ratio was calculated by the following calculation formula.

### (4) Calculation Formula of the Herbicidal Ratio

Herbicidal ratio (%)=(the fresh grass mass at an aerial part on untreated area−the fresh grass mass at an aerial part on treated area)/(the fresh grass mass at an aerial part on untreated area)×100

Each of the compounds A-1, A-3, A-4, A-5, A-7 and A-8 was sprayed at a spray amount of 250 g per hectare. The herbicidal ratio of each compound against Digitaria ciliaris was 100%.

Each of the compounds A-1, A-2, A-3, A-4, A-5, A-7, A-8 and A-9 was sprayed at a spray amount of 250 g per hectare. The herbicidal ratio of each compound against Matricaria chamomilla was 100%.

Each of the compounds A-1, A-2, A-3, A-4, A-5, A-7 and A-9 was sprayed at a spray amount of 250 g per hectare. The herbicidal ratio of each compound against Avena sativa was 100%.

Each of the compounds A-1, A-3, A-5, A-7 and A-9 was sprayed at a spray amount of 250 g per hectare. The herbicidal ratio of each compound against Setaria faberi was 100%.

Each of the compounds A-1, A-3, A-4, A-5, A-7, A-8 and A-9 was sprayed at a spray amount of 250 g per hectare. The herbicidal ratio of each compound against Abutilon theophrasti was 90% or more.

Each of the compounds A-1, A-3, A-4, A-5, A-7, A-8 and A-9 was sprayed at a spray amount of 250 g per hectare. The herbicidal ratio of each compound against Amaranthus retroflexus was 100%.

### (Test Example 2) Soil Treatment Test

### (1) Preparation of Test Emulsions

A test emulsion was prepared by the same method as that in Test example 1.

### (2) Soil Treatment

70 cm² pots were filled with soil, and then seeds of Digitaria ciliaris and Amaranthus retroflexus were sown on the surface layer of each of the pots, respectively, followed by lightly covering with soil. On the next day, the above-mentioned test emulsion was diluted to obtain a predetermined active ingredient amount, and then was sprayed onto the soil surface at a spray volume of 2860 L per hectare using a small sprayer.

### (3) Evaluation

Four weeks after, the fresh grass mass at an aerial part on an untreated area or a treated area was measured per weed, and the herbicidal ratio was calculated by the following calculation formula.

### (4) Calculation Formula of the Herbicidal Ratio

Herbicidal ratio (%)=(the fresh grass mass at an aerial part on untreated area−the fresh grass mass at an aerial part on treated area)/(the fresh grass mass at an aerial part on untreated area)×100

The compounds A-1 and A-4 were sprayed at a spray amount of 63 g or 31 g per hectare, respectively.

In addition, the following compounds (A) to (C) disclosed in Patent Document 1 were sprayed in a similar manner.

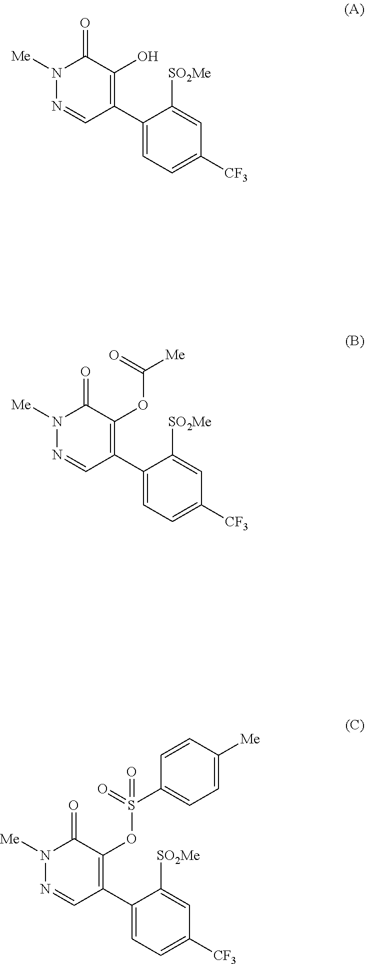 [SMILES: Cn1ncc(-c2ccc(C(F)(F)F)cc2S(C)(=O)=O)c(O)c1=O ; CC(=O)Oc1c(-c2ccc(C(F)(F)F)cc2S(C)(=O)=O)cnn(C)c1=O ; Cc1ccc(S(=O)(=O)Oc2c(-c3ccc(C(F)(F)F)cc3S(C)(=O)=O)cnn(C)c2=O)cc1]

The herbicidal ratio of each compound is shown in Table 2.

TABLE 2

| | | Herbicidal ratio | |
|---|---|---|---|
| | | | Amaranthus |
| Compound No. | Spray amount | Digitaria ciliaris | retroflexus |
| A-1 | 63 g/ha | 100% | 100% |
| | 31 g/ha | 100% | 100% |
| A-4 | 63 g/ha | 100% | 100% |
| (A) | 63 g/ha | 0% | 0% |
| | 31 g/ha | 0% | 0% |
| (B) | 63 g/ha | 30% | 0% |
| | 31 g/ha | 0% | 0% |
| (C) | 63 g/ha | 70% | 0% |
| | 31 g/ha | 40% | 0% |

It was confirmed that the herbicidal effects of the compound according to the present invention applied by soil treatment were significantly improved in comparison with those of the compound disclosed in Patent Document 1.

### INDUSTRIAL APPLICABILITY

The pyridazine compound according to the present invention exhibits reliable weed control effects even at a low dose, causes fewer harmful effects on crops, and has high environmental safety, and therefore is useful as an active ingredient of a herbicide. The herbicide according to the present invention can be used safely to control weeds in the cultivation of agricultural or horticultural crops.

1. A compound of formula (I) or a salt thereof: 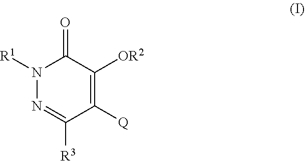
in the formula (I),
R¹ represents a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group,
R² represents a group of R^{a}—CO—O—CR^{b}₂— or a group of R^{a}O—CO—O—CR^{b}₂—,
R^{a} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,
R^{b} each independently represents a hydrogen atom or a substituted or unsubstituted C1-6 alkyl group,
R³ represents a hydrogen atom, a substituted or unsubstituted C1-6 alkyl group, a substituted or unsubstituted C2-6 alkenyl group, a substituted or unsubstituted C2-6 alkynyl group, or a substituted or unsubstituted C3-6 cycloalkyl group, and
Q represents a substituted or unsubstituted phenyl group, or a substituted or unsubstituted naphthyl group.

2. The compound or the salt according to claim 1, wherein
a substituent of the phenyl group or the naphthyl group as Q is at least one selected from the group consisting of halogeno groups, substituted or unsubstituted C1-6 alkyl groups, substituted or unsubstituted C2-6 alkenyl groups, substituted or unsubstituted C2-6 alkynyl groups, a hydroxy group, substituted or unsubstituted C1-6 alkoxy groups, substituted or unsubstituted C2-6 alkenyloxy groups, substituted or unsubstituted C2-6 alkynyloxy groups, substituted or unsubstituted C1-6 alkylthio groups, substituted or unsubstituted C1-6 alkylsulfinyl groups, substituted or unsubstituted C1-6 alkylsulfonyl groups, substituted or unsubstituted C3-6 cycloalkyl groups, substituted or unsubstituted C3-6 cycloalkyloxy groups, substituted or unsubstituted phenyl groups, substituted or unsubstituted naphthyl groups, substituted or unsubstituted phenoxy groups, substituted or unsubstituted naphthoxy groups, substituted or unsubstituted phenylthio groups, substituted or unsubstituted naphthylthio groups, substituted or unsubstituted phenylsulfinyl groups, substituted or unsubstituted naphthylsulfinyl groups, substituted or unsubstituted phenylsulfonyl groups, substituted or unsubstituted naphthylsulfonyl groups, substituted or unsubstituted 5-membered or 6-membered heterocyclyl groups, substituted or unsubstituted 5-membered or 6-membered heterocyclyloxy groups, substituted or unsubstituted 5-membered or 6-membered heterocyclylthio groups, substituted or unsubstituted 5-membered or 6-membered heterocyclylsulfinyl groups, substituted or unsubstituted 5-membered or 6-membered heterocyclylsulfonyl groups, a nitro group, a cyano group, groups of R^{c}—CO—, a carboxy group, groups of R^{d}—O—CO—, groups of R^{e}R^{f}N—, groups of R^{e}R^{f}N—CO—, groups of R^{e}R^{f}N—SO₂—, groups of R^{c}—CO—O—, groups of R^{c}—CO—NH—, groups of R^{d}—SO₂—NH—, groups of R^{d}—O—CO—O—, groups of R^{d}—O—CO—NH—, groups of R^{e}R^{f}N—CO—O—, and groups of R^{e}R^{f}N—CO—NH—;
R^{c} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,
R^{d} each independently represents a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,
R^{e} each independently represents a hydrogen atom, a substituted or unsubstituted C1-6 alkyl group, a substituted or unsubstituted C2-6 alkenyl group, a substituted or unsubstituted C2-6 alkynyl group, a substituted or unsubstituted C3-6 cycloalkyl group, a substituted or unsubstituted phenyl group, a substituted or unsubstituted naphthyl group, or a substituted or unsubstituted 5-membered or 6-membered heterocyclyl group,
R^{f} each independently represents a hydrogen atom, a substituted or unsubstituted C1-6 alkyl group, a substituted or unsubstituted phenyl group, or a substituted or unsubstituted naphthyl group,
R^{e} and R^{f} may form a divalent organic group together, and
two substituents on the phenyl group or the naphthyl group as Q may be bonded together with atoms constituting the phenyl group or the naphthyl group bonded thereby to form a substituted or unsubstituted C5-6 non-aromatic carbon ring, a substituted or unsubstituted nitrogen-containing hetero ring, a substituted or unsubstituted oxygen-containing hetero ring, or a substituted or unsubstituted oxygen and nitrogen-containing hetero ring.

3. A herbicide comprising at least one selected from the group consisting of a compound and a salt thereof of claim 1 as an active ingredient thereof.

4. A method for controlling monocot and/or dicot weeds on useful plants, comprising a step of applying a compound or a salt thereof of claim 1, or a herbicide comprising the compound on the weeds, the useful plants, and/or places on which the weeds and/or the useful plants exist.
